(19) United States
(12) Patent Application Publication
(10) Pub. No.: US 2023/0363685 A1
(43) Pub. Date: Nov. 16, 2023

(54) WEARABLE SYSTEM FOR MUSCLE ACTIVITY SENSING AND FEEDBACK

(71) Applicant: The Johns Hopkins University, Baltimore, MD (US)

(72) Inventors: Korine A. Ohiri, Arlington, VA (US); Luke J. Currano, Columbia, MD (US); Luke E. Osborn, Baltimore, MD (US); Eric Q. Nguyen, Hanover, MD (US); Christopher J. Dohopolski, Baltimore, MD (US)

(21) Appl. No.: 18/224,151

(22) Filed: Jul. 20, 2023

Related U.S. Application Data
(63) Continuation-in-part of application No. 17/093,799, filed on Nov. 10, 2020.
(60) Provisional application No. 63/391,979, filed on Jul. 25, 2022.
(60) Provisional application No. 62/961,737, filed on Jan. 16, 2020.

(51) Int. Cl. A61B 5/296; A61B 5/256; A61B 5/00

(52) U.S. Cl. CPC A61B 5/296; A61B 5/256; A61B 5/4519; A61B 5/7405; A61B 5/742; A61B 2562/0209

(57) ABSTRACT

A system for muscle activity sensing and feedback includes a base textile, an electrode coupled to the base textile, a sensor coupled to the base textile, a controller coupled to the base textile, and a feedback element coupled to the base textile. The feedback element is in communication with the controller. The feedback element receives a feedback signal from the controller and imparts feedback to a user based on an electrical signal from the electrode and/or a sensor signal from the sensor.

### CROSS-REFERENCE TO RELATED APPLICATIONS

This application claims priority to and the benefit of prior-filed, co-pending U.S. Provisional Application No. 63/391,979 filed on Jul. 25, 2022, and is a continuation-in-part of prior-filed, co-pending U.S. Nonprovisional Application No. 17/093,799 filed on Nov. 10, 2020, which claims priority to and the benefit of prior-filed U.S. Provisional Application No. 62/961,737 filed on Jan. 16, 2020, the entire contents of each of which are hereby incorporated herein by reference.

### STATEMENT OF GOVERNMENTAL INTEREST

This invention was made with Government support under contract number HU00012120074 awarded by the Uniformed Services University of the Health Sciences (USU). The Government has certain rights in the invention.

### TECHNICAL FIELD

Example, non-limiting embodiments relate generally to sensing and feedback systems for wearable muscle activity sensors and, more particularly, relate to tactile/haptic feedback integrated into physiological sensing devices and garments.

### BACKGROUND

Muscle activity detection has proven useful in a number of contexts, particularly in the areas of exercise science and in detecting neuromuscular abnormalities. One manner of detecting muscle activity, known as electromyography, detects electrical signals that travel from the brain via the nervous system to control the muscle. These signals are sometimes detected by a sensor that is inserted through the skin and into the muscle, such as with a pin that is connected to a wire. This approach is invasive and can be uncomfortable for the individual, especially when the individual needs to wear the sensor for an extended period of time and/or while moving around with the sensor inserted. Also, the insertion of a sensor into the muscle itself may not be practical in many contexts, particularly when tracking signals and associated movements during non-medical, standard or day-to-day scenarios, such as while a user of the sensor is at home or while exercising. An alternative to invasive electromyography is surface electromyography, typically performed with electrodes, having a conductive gel applied to them, being adhered to the skin with an adhesive. However, these electrodes are inconvenient and uncomfortable, especially when used over areas of the skin with hair on them. Additionally, the conductive gel dries out over time, degrading or blocking the electrode’s ability to detect the signal.

As a result, there is an ongoing need for further development of such sensors and their associated systems, such as supplementing and improving these sensors, and the systems or garments that they are employed in, with feedback that can be provided to a user or wearer of the sensors or garments.

### BRIEF SUMMARY

In one example embodiment, a system for muscle activity sensing and feedback includes a base textile, an electrode coupled to the base textile, a sensor coupled to the base textile, a controller coupled to the base textile, and a feedback element coupled to the base textile. The feedback element is in communication with the controller. The feedback element receives a feedback signal from the controller and imparts feedback to a user based on an electrical signal from the electrode and/or a sensor signal from the sensor.

In another example embodiment, a system for muscle activity sensing and feedback includes a base textile configured to apply a compression force against a dermal surface of a user, an electrode coupled to the base textile and configured to receive an electrical signal associated with muscle activity of the user, a sensor coupled to the base textile and configured to sense a parameter associated with a condition of either the base textile or an environment near the base textile. The sensor is also configured to generate a sensor signal based on the sensed parameter. The system also includes a controller coupled to the base textile and configured to receive the electrical signal from the electrode and/or the sensor signal from the sensor. The controller is also configured to analyze the electric signal and/or the sensor signal, and to generate a feedback signal. The system also includes a feedback element coupled to the base textile and in communication with the controller. The feedback element is configured to receive the feedback signal from the controller and to impart feedback to the user based on the electrical signal from the electrode and/or the sensor signal from the sensor.

### BRIEF DESCRIPTION OF THE DRAWINGS

Having thus described some non-limiting, example embodiments in general terms, reference will now be made to the accompanying drawings, which are not necessarily drawn to scale, and wherein:

FIG. 1 illustrates an example muscle activity sensor system according to some example embodiments;

FIG. 2 illustrates a cross-section view of a muscle activity electrode according to some example embodiments;

FIGS. 3A to 3C illustrate top views of muscle activity electrodes with different options for feedthroughs according to some example embodiments;

FIG. 4 illustrates a pair of nested serpentine interconnects according to some example embodiments;

FIGS. 5A and 5B illustrate components of a twisted pair interconnect according to some example embodiments;

FIG. 5C illustrates an assembled twisted pair interconnect according to some example embodiments;

FIG. 6 illustrates a collection of interconnects that extend to a common junction according to some example embodiments;

FIG. 7 illustrates a tab assembly according to some example embodiments;

FIG. 8A illustrates cross-section view of a tab assembly according to some example embodiments;

FIG. 8B illustrates a cross-section view of a tab assembly engaged with a circuit board socket according to some example embodiments;

FIG. 9. illustrates a cross-section view of a haptic feedback element according to some example embodiments;

FIG. 10 illustrates a cross-section view of another haptic feedback element according to some example embodiments;

FIG. 11A illustrates a wireless controller according to some example embodiments;

FIG. 11B illustrates a muscle activity sensor system disposed in a garment in according to some example embodiments;

FIG. 12 illustrates a block diagram of a wireless controller according to some example embodiments; and

FIG. 13 illustrates a flow of information in a wireless controller and/or external client device according to some example embodiments.

### DETAILED DESCRIPTION

Some non-limiting, example embodiments will be described more fully hereinafter with reference to the accompanying drawings, in which some, but not all example embodiments are shown. Indeed, the examples described and pictured herein should not be construed as being limiting as to the scope, applicability, or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that yields a true result whenever one or more of its operands are true. As used herein, coupling should be understood to relate to direct or indirect connection that, in either case, enables functional interconnection of components that are coupled to each other.

Wearable muscle activity electrodes, sensors, feedback components, and associated methods, are described herein. In an example embodiment, The muscle activity sensors and associated electrodes may collect electromyography (EMG) signals to assess muscle health and the nerves that control the muscles. Such sensors may be a type of physiological sensor, such as a muscle activity sensor, which may include one or more muscle activity electrodes, and may be integrated into a base textile such as sleeve, shirt, pants, or other type of garment. The base textile may be formed of or from a textile with elastic properties, to provide a compression force against the skin or dermal surface of a user wearing the base textile. A muscle activity electrode that is coupled to the base textile may be pressed against the dermal surface at a location adjacent to a muscle. The electrode may include a sensor layer that is formed from or of a conductive textile, for example, and may receive electrical signals (e.g., EMG signals) originating from and associated with muscle activity of the user/wearer. The electrode may also be configured to deliver the EMG signals detected in the muscle to an interconnect that is also coupled to the base textile. The interconnect delivers the EMG signals from the electrode to a junction that is configured to be coupled to a processing device. The interconnect may therefore extend on the base textile across a length of the base textile and may therefore be configured to move with the base textile when, for example, a user moves (e.g., bends at a joint). To reduce the mechanical forces applied to the interconnect (such as stresses and strains on the base textile and, as a result, the interconnect due to movement of the user) the interconnect may also be formed of or from a conductive textile and may be shaped in a serpentine pattern, which limits the stresses and strains on the interconnect, but also minimizes the variation in electrical resistance of the interconnect as a result of shape changes from the user’s movements. The interconnect may be affixed to the exterior surface of the base textile and travel along the exterior surface of the base textile in a serpentine pattern to a junction. Further, a number of interconnects may terminate at the junction area, with each interconnect having a respective interconnect junction contact. Additionally, two interconnects may be formed as a nested serpentine pair or as twisted pair, as further described herein. To provide for connectivity between the interconnect junction contacts and a circuit board, a tab assembly may be may be affixed to the interconnect junction contacts. The tab assembly may include a conductive element affixed to a support layer (formed of or from a polyimide film, for example) to form a plug. The plug of the tab assembly may be received into a circuit board socket to form electrical connections to each of the interconnect junction contacts.

While some example embodiments describe herein are directed to applications involving muscle activity sensing, it is contemplated that example embodiments may be implemented to detect other electrical signals emanating from the body. For example, some example embodiments may be employed in the context of electrophysiological sensing, such as EEG (electroencephalogram) sensing of the brain, ECG/EKG (electrocardiogram/elektrokardiogramm) sensing of the heart, bioimpedance of the body or parts of the body, or galvanic skin response (GSR). Such example embodiments may differ, for example, in the placement of the electrodes relative to the sensing target (e.g., brain, heart, etc.).

Additionally, example embodiments may be configured to include feedback capabilities, to provide sensory (e.g., haptic) feedback to a user or wearer of the base textile having the muscle activity sensors and associated electrodes. To provide the feedback capability, feedback connectors may be affixed to the base textile to connect feedback elements disposed on or in the base textile to the junction.

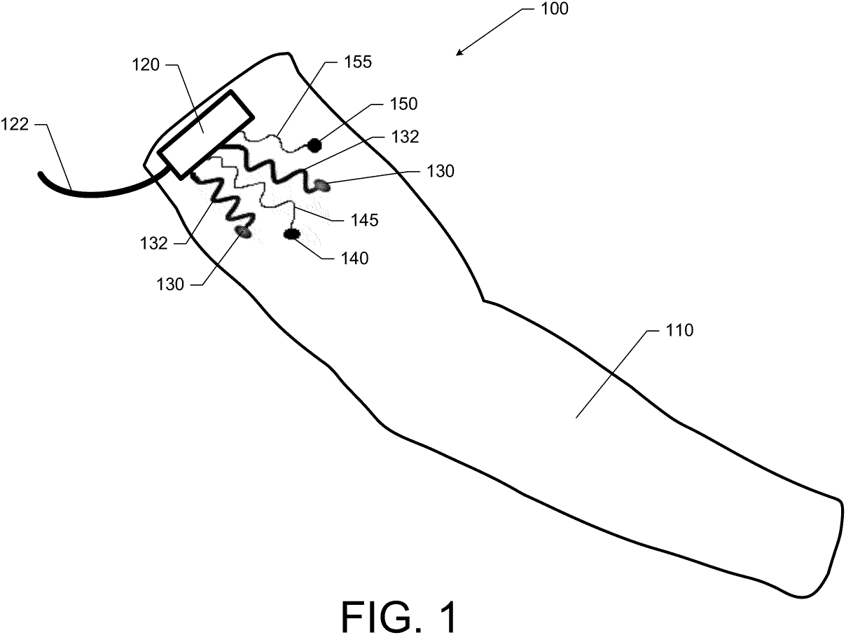

Having described some example embodiments in general terms, FIG. 1 illustrates an example muscle activity sensor system 100. As mentioned above, the muscle activity sensor system 100 may be constructed on a base textile 110. Although the base textile 110 may take any form of clothing that may be worn or applied to a user’s body (e.g., shirt, shorts, pants, calf sleeve or band, arm sleeve or band, or the like), the base textile 110 of FIG. 1 is shown as an arm sleeve. According to some example embodiments, the base textile 110 may include a cuff that, for example, may be secured in place using VELCRO® or other hook and loop fasteners. According to some example embodiments, the base textile 110 may have an elastic property and may therefore be configured to apply a compression force against the skin or dermal surface of the user. As such, the base textile 110 may have a dermal side and an external side, with the external side being shown in FIG. 1. The base textile 110 may be formed of or from, or include spandex, elastane, LYCRA®, or another textile including, for example, polyether-polyurea copolymer fibers or other elastic, synthetic fiber-based textiles or materials.

The muscle activity sensor 100 system may also include electrodes 130 (e.g., muscle activity electrodes 130). More specifically, an electrode 130 according to an example embodiment may be constructed such that the base textile 110 supports and forms a component of the electrode 130. The electrode 130 may be configured to detect and receive electrical signals, e.g., EMG signals, emitted by a muscle during a muscle movement, for delivery to, and analysis by, processing circuitry. The electrode 130 may be coupled to the base textile 110 in variety of ways to place the electrode 130 in close proximity to or adjacent to a target muscle. For example, the electrode 130 may be coupled to the base textile 110 by being sewn or embroidered onto the base textile 110. Alternatively, the electrode 130 may be affixed to the base textile 110 via an adhesive (e.g., hot-melt adhesive), via lamination (e.g., heat lamination), or the like. According to some example embodiments, the electrode 130 may have a component that is disposed on the internal, dermal side of the base textile 110 such that the component may be in direct contact with the skin or dermal surface of the user. Further, the electrodes 130 may be located, based on the type of garment formed by the base textile, in a position in close proximity to or adjacent to a target muscle of the user. As shown in FIG. 1, the electrodes 130 are located proximate or adjacent to the bicep and tricep muscles, for example. According to some example embodiments, to monitor a given muscle, two electrodes 130 may be used and the signal differential between the EMG signals received by the respective electrodes 130 may be used for muscle response monitoring, electromyography applications, or the like.

The electrodes 130 may deliver the received EMG signals from the muscles to an interconnect 132. An interconnect 132 may also be coupled to the base textile 110, and may be configured to the deliver the EMG signal received by a respective electrode 130 to, for example, a junction area. For coupling, similar to the electrode 130, the interconnect 132 may be coupled to the base textile 110 by being sewn to, or embroidered on, the base textile 110. Alternatively, the interconnect 132 may be affixed to the base textile 110 via an adhesive (e.g., hot-melt adhesive), via lamination (e.g., heat lamination), or the like. Further, the interconnect 132 may also be reinforced and/or electrically insulated by performing a potting operation on the interconnect 132 by curing, for example, a urethane layer (e.g., at room temperature) or heat laminating a urethane layer to the interconnect 132. The interconnect 132 may be coupled to either the dermal side or the exterior side of the base textile 110. As such, the interconnect 132 may be the component that transmits the EMG signal of the electrode 130 from a position proximate to or adjacent to a muscle to a junction area for provision to processing circuitry. According to some example embodiments, the interconnect 132 may be formed of or from a conductive textile to support conduction of the EMG signals. For example, the interconnect 132, as a conductive textile, may be formed of or from, for example, synthetic elastane fibers with a conductive coating or synthetic elastane fibers woven with conductive fibers. According to some example embodiments, a spandex, LYCRA®, or the like coated with metal (e.g., silver) may be used for the interconnect 132. Accordingly, the shape of the interconnect 132 may be formed via laser cutting or die or stamp cutting of the conductive textile. According to some example embodiments, the interconnect 132 may be formed by other materials such as, for example, conductive paints or inks applied to the base textile 110.

Additionally, to limit the stresses and strains placed on an interconnect 132, the interconnect 132 may be formed in a variety of shapes. For example, the interconnect 132 may take a non-linear shape. In this regard, a portion of the interconnect 132 may take a serpentine shape, which may include a sinusoidal shape, a zig-zag shape, or the like. Such shapes may be configured to reduce the stress and strain on the interconnect 132, which may affect the electrical resistance across the interconnect 132. Because, for example, a serpentine shape is not subjected to high stresses or strains when the base textile 110 is moved (e.g., stretched by movement of the user), the electrical resistance across the interconnect 132 may be remain within a threshold range (e.g., below about 1 kilohm) and thus relatively constant during movement of the user’s body. As such, electrical (e.g., EMG) signal measurements from the muscle can be reliable, even when the user is moving.

The interconnects 132 may terminate at a junction area where connections may be made to a tab assembly as described in greater detail below. The tab assembly may form or include a plug that may be received into a circuit board socket of a circuit board 120. As such, the circuit board 120 may be electrically connected to the interconnects 132 via the tab assembly and the circuit board socket. The circuit board 120 may include processing circuitry for analyzing signals provided by the electrodes 130 and/or sensors 150, and for generating signals to control feedback elements 140 based on the signals from the electrodes 130 and/or sensors 150, as will be described in greater detail below.

Still referring to FIG. 1, the muscle activity sensor system 100 according to an example embodiment includes an integrated sensory feedback capability. To that end, the muscle activity sensor system 100 includes the feedback elements 140 connected to the circuit board 120 by feedback connectors 145. In an example embodiment, the feedback elements 140 and the feedback connectors 145 are disposed on the base textile 110, to provide feedback to a user or wearer of the base textile 110. As described in greater detail below, the feedback includes, but is not limited to, haptic feedback (e.g., mechanical feedback to provide a sense of touch by creating sensations using vibration, pressure, force, or other movement), as well as thermal/thermotactile feedback/stimulation (e.g., heating or cooling), and other types of feedback or sensations (audio, visual, electrical/electrostatic, etc.). In an example embodiment, the circuit board 120 is a controller 120 and, more specifically, is a wireless controller 120, as will also be described in greater detail below. When the circuit board/controller 120 is wireless, the wired connection 122 shown in FIG. 1 is not needed and may be removed, or may be replaced with an antenna 122 for the wireless board/controller 120.

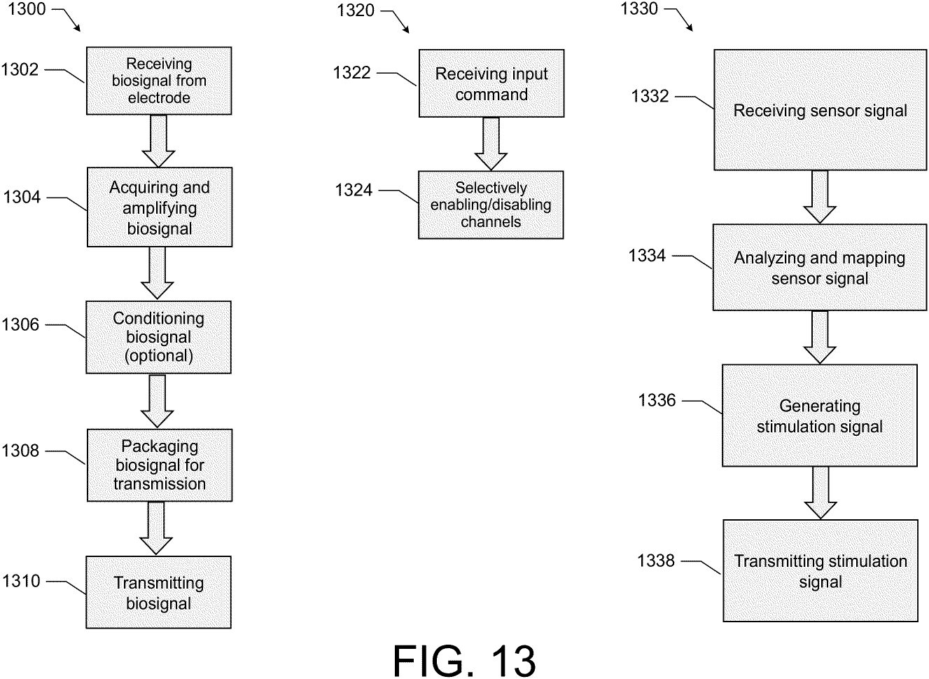

The muscle activity sensor system 100 according to an example embodiment may also include one or more of the sensors 150, the outputs of which are transmitted to the controller 120 via sensor connections 155 and are used to generate stimulation signals to drive the feedback elements 140, as will be described in greater detail below with reference to FIG. 13. The sensors 150 may include various types of sensors, such as thermal, pressure, force, moisture, oxygen, electromagnetic (EM) field, magnetic, chemical, ultraviolet (UV), infrared (IR), radiation, and other types of sensors. Signals from such sensors may be used in conjunction with any of the types of feedback and feedback elements to alert the user or wearer of a garment including the system 100 to environmental conditions or hazards. For example, a temperature sensor 150 may sense a temperature on or near the garment and transmit a corresponding sensor signal to the controller 120, which then transmits a resulting stimulation feedback signal to one or more of the feedback elements 140 causing the feedback element to generate heat (or cold) based on the temperature (or a change thereof) sensed by the sensor 150. In a similar manner, a humidity or water/moisture sensor 150 could be used to alert the user to the presence (or absence) of moisture or water. As another example, an oxygen sensor 150 could be used to alert a user of low oxygen levels in the air, while a radiation sensor 150 would alert the user to the presence of (or high levels of) radiation, etc. Similarly, UV sensors could be used to alert users to the presence of certain chemicals, and IR sensors 150 could be used to aid users in locating objects by detecting temperatures of certain objects relative to the background or other objects.

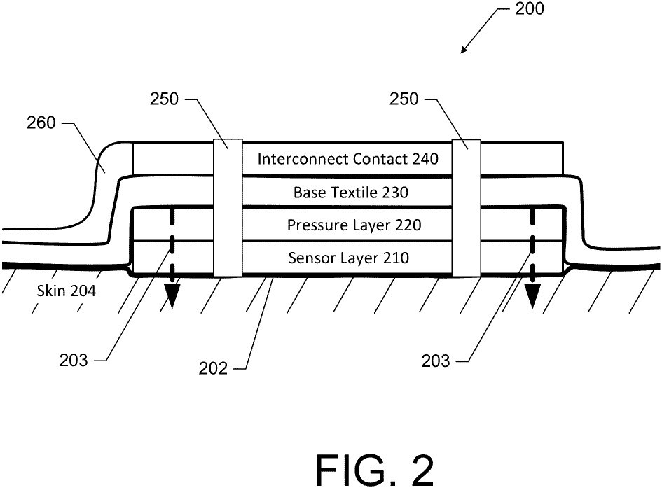

Now referring to FIG. 2, a cross-section view of a single muscle activity electrode 200 (e.g., one of the muscle activity electrodes 130 shown in FIG. 1) according to some example embodiments is shown. The electrode 200 may be the same or similar to the electrode 130 shown in FIG. 1 in operation and structure. In this regard, the electrode 200 may include a sensor layer 210, a pressure layer 220, a base textile 230, an interconnect contact 240, and a feedthrough element 250. The electrode 200 may be positioned to be in direct contact to the skin 204 of the user and, more specifically, to the dermal surface 202 of the skin 204 of the user. As described above, the base textile 230, which may be the same or similar to the base textile 110 of FIG. 1 in function and structure, may serve as a substrate for the electrode 200 and may also operate to apply a compression force (as indicated by the arrows 203) to press the electrode 200 against the dermal surface 202.

The sensor layer 210 may be configured to be in contact with the dermal surface 202 to receive electrical (e.g., EMG) signals associated with an underlying muscle. Further, the sensor layer 210 may be formed of or from a conductive textile. In this regard, the sensor layer 210 may be formed of or from, for example, a conductive textile including synthetic elastane fibers with a conductive coating or synthetic elastane fibers woven with conductive fibers. According to some example embodiments, a silver (metal) spandex, LYCRA®, elastane, or the like may be used for the sensor layer 210. Further according to some example embodiments, the sensor layer 210 may be formed of or from other types of conductive textiles, such as, polyester or another textile coated with poly(3,4-ethylenedioxythiophene) polystyrene sulfonate (PEDOT:PSS). According to some example embodiments, combinations of, for example, conductive textiles may be used, such as combining silver LYCRA® with PEDOT:PSS-coated polyester in layers to form the sensor layer 210. Alternatively, according to some example embodiments, the sensor layer 210 may be formed by other materials such as, for example, conductive paints or inks. As a conductive textile, the sensor layer 210 may be configured to absorb, hold, or wick moisture to improve the electrical contact (e.g., reduce electrical resistance) between the sensor layer 210 and the dermal surface 202. In this manner, the inclusion of moisture (e.g., water, sweat, etc.) to the sensor layer 210 may increase the electrode 200′s ability to reliably detect the electrical (e.g., EMG) signals associated with the target muscle.

In some example embodiments, the sensor layer 210 may be applied directly to the base textile 230. However, according to some example embodiments, a pressure layer 220 may be disposed between the sensor layer 210 and the base textile 230, as shown in FIG. 2. The pressure layer 220 may be configured to add thickness between the dermal layer contacting surface of the sensor layer 210 and the base textile 230. By doing so, the compression force generated by the base textile 230 on the dermal surface 202 may be increased to ensure reliable contact between the sensor layer 210 and the dermal surface 202. According to some example embodiments, the pressure layer 220 may be formed or include a foam material.

With the pressure layer 220 and the sensor layer 210 being disposed on the dermal side of the base textile 230, the electrode 200 may also include an interconnect contact 240 disposed on the external side of the base textile 230. The interconnect contact 240 may be a portion of or a connection point to an interconnect 260, which may be the same or similar to the interconnect 132 of FIG. 1 in function and structure. As such, the interconnect contact 240 may be formed of or from the same or similar materials as the interconnect 132, and therefore, according to some example embodiments, the interconnect contact 240 may be formed of or from a conductive textile or a conductive paint or ink.

To electrically connect the interconnect contact 240 to the sensor layer 210, the electrode 200 may include a feedthrough element 250. The feedthrough element 250 may be formed of or from a conductive material (e.g., metal) that pierces through the interconnect contact 240 and the base textile 230 to come into physical and electrical contact with the sensor layer 210. According to some example embodiments, in addition to forming an electrical connection between the interconnect contact 240 and the sensor layer 210, the feedthrough element 250 may mechanically couple the sensor layer 210, the pressure layer 220, and the interconnect contact 240 to the base textile 230. According to some example embodiments, the feedthrough element 250 may be a metallic snap or grommet. Alternatively, according to some example embodiments, the feedthrough element 250 may be formed by a conductive thread or yarn.

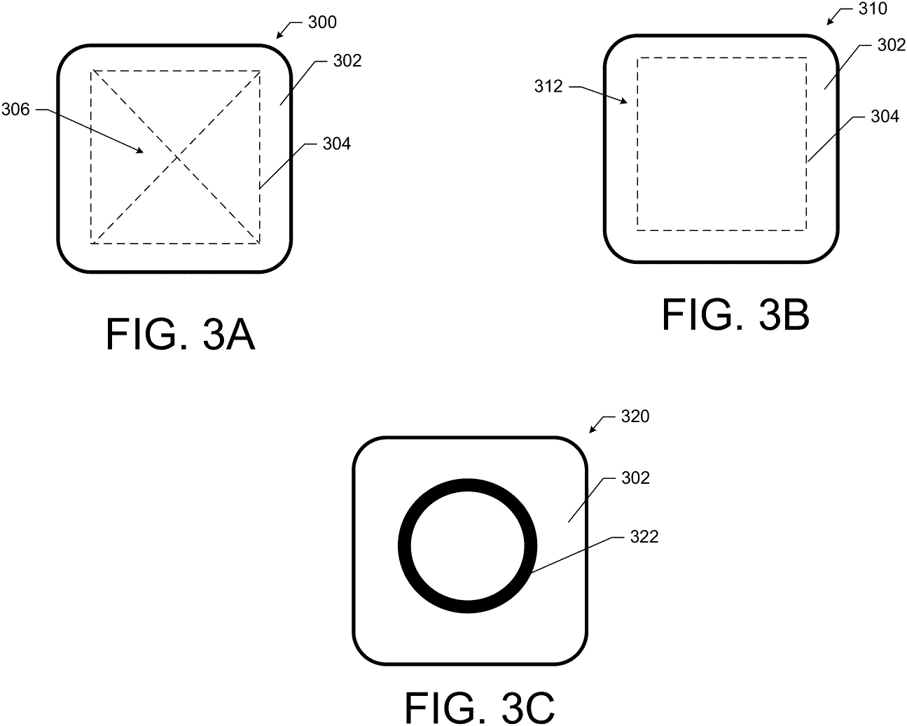

FIGS. 3A to 3C illustrate top views of various example muscle activity electrodes with different options for feedthroughs according to some example embodiments. In this regard, FIG. 3A illustrates an example electrode 300 including a interconnect contact 302 and a feedthrough element 306. The feedthrough element 306 may be formed by, for example, a conductive thread 304 sewn into the electrode 300. The conductive thread 304 may be sewn around the edges of the generally square interconnect contact 302 and in an x-pattern through the center of the electrode 300 to form the feedthrough element 306.

FIG. 3B illustrates an example electrode 310 including a interconnect contact 302 and a feedthrough element 312. The feedthrough element 312 may be formed by conductive thread 304 sewn into the electrode 310. The conductive thread 304 may be sewn around the edges of the generally square interconnect contact 302 to form the feedthrough element 312. While the interconnect contact 302 is shown as being generally square, the contact may take a variety of different shapes (e.g., circle, oval, rectangle, triangle, ring or donut, bow-tie, or the like). Further, regardless of the shape of the interconnect contact 302 (and accordingly, the shape of the electrode) the conductive thread 304 may be sewn, for example, around the edges of the interconnect contact 302.

Alternatively, FIG. 3C illustrates an example electrode 320 including a interconnect contact 302 and a feedthrough element 322. The feedthrough element 322 may be formed by a conductive snap device or a grommet device that has components that are forced into the electrode 310 and secured together. The snap or grommet may be pressed into the electrode 320 and the interconnect contact 302 to form the feedthrough element 322.

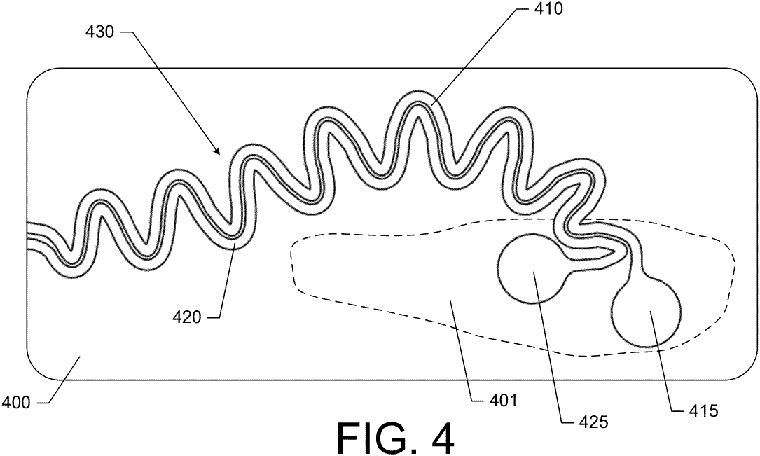

FIG. 4 illustrates a sensor arrangement that includes an interconnect pair 430 of nested serpentine interconnects 410 and 420 connected to respective electrodes 415 and 425 affixed to a base textile 400 proximate to a target muscle 401 according to some example embodiments. As shown, the interconnects 410 and 420 follow similar paths from the electrodes 415 and 425. Further, the interconnect pair 430 is configured to allow the serpentine interconnects 410 and 420 to move together due to their close proximity and, therefore, any effect of stress and strain on the electrical resistance of the electrodes 415 and 425 may be the same or similar. Each interconnect 410 and 420 may include segments or portions that are sinusoidal in shape or structure. However, some bending and curvatures may be introduced to the sinusoidal structures. As a serpentine structure, the interconnects 410 and 420 may include a portion with a sinusoidal structure, but may, in some example embodiments, also include a components that introduce bends and turns for positioning the interconnects 410 and 420 between the target muscle 401 and the junction area (not shown). Additionally, the portion of the interconnect pair 430 that is structured as a nested sinusoid or components of a nested sinusoid include segments where one of the interconnects is subjected to a tighter turn (on an interior of the turn) and the other of the interconnects is subjected to wider turn (on the exterior of the turn). As the nested sinusoidal shape travels across the base textile 400, the interconnect that is subjected to a tighter turn is subsequently subjected to a wider turn and vice versa. Because of this relationship within the interconnect pair 430, the lengths of the interconnects may be the same or similar and may be subjected to the same or similar strain and stress forces when the base textile 400 moves or stretches.

Additionally, because the interconnect pair 430 maintains adjacency across a substantial portion of the lengths of the serpentine interconnects 410 and 420, an inductive coupling may occur between the interconnects 410 and 420. Such inductive coupling can operate to reduce noise by canceling some effects of noise that may introduced to the electrical signals being delivered by the interconnects 410 and 420.

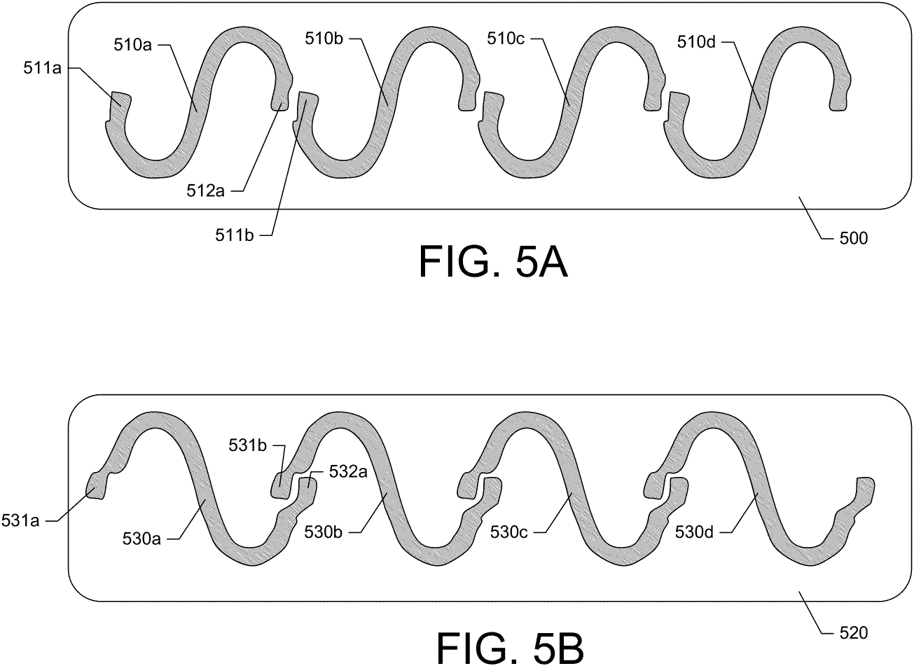

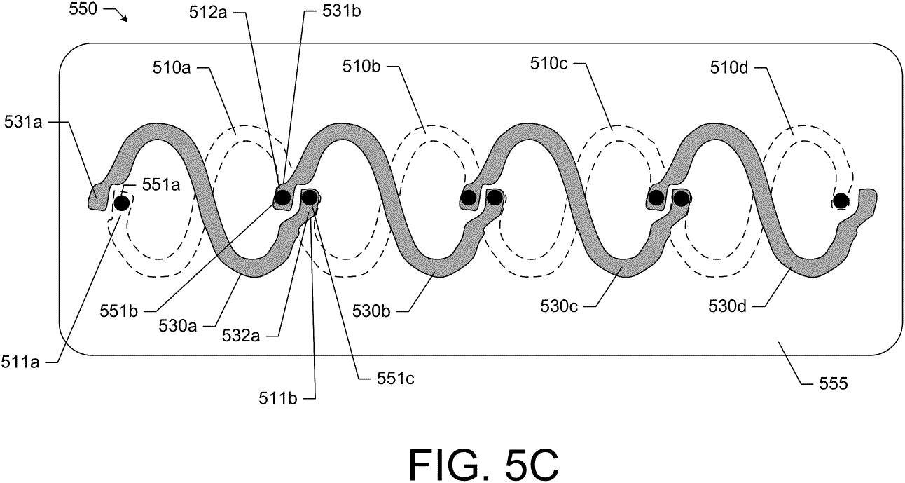

In some instances, to increase the inductive coupling that may occur between two interconnects (such as the interconnects 410 and 420 described above and shown in FIG. 3C), to further reduce noise, for example, an alternative twisted pair configuration for interconnects may be employed, as shown in FIGS. 5A to 5C. In this regard, conductive segments of piecewise sinusoid shapes (formed, for example, using the conductive textiles described herein) may be formed on a substrate. Specifically, with reference to FIG. 5A, a first substrate 500 may support repeating piecewise sinusoid segments 510a, 510b, 510c, and 510d. Individually, each of the piecewise sinusoidal segments 510a, 510b, 510c, and 510d may take the form of a single period (cycle) of a sinusoid and, again, the sinusoidal shape may be used to reduce the stress and strain on the segments when applied to a movable base textile. With reference to a single, particular piecewise sinusoidal segment 510a as an example, the piecewise sinusoidal segment 510a may include a first end contact 511a disposed at one end of the piecewise sinusoidal segment 510a, and a second end contact 512a disposed at the other end of the piecewise sinusoidal segment 510a. As described in greater detail below, the piecewise sinusoidal segments 510a, 510b, 510c, and 510d may, in combination, form one side layer of a twisted pair of interconnects.

Similarly, as shown in FIG. 5B, a second substrate 520 may support repeating piecewise sinusoidal segments 530a, 530b, 530c, and 530d. Individually, each piecewise sinusoidal segment 530a, 530b, 530c, and 530d may take the form of a single period of a sinusoid and, again, the sinusoidal shape may be used to reduce the stress and strain on the segments when applied to a movable base textile. With reference to the single piecewise sinusoidal segment 530a as an example, the piecewise sinusoidal segment 530a may include a first end contact 531a disposed at one end of the piecewise sinusoidal segment 530a, and a second end contact 532a disposed at the other end of the piecewise sinusoidal segment 530a. As described in greater detail below, the piecewise sinusoidal segments 530a, 530b, 530c, and 530d may form another side layer of a twisted pair of interconnects. Of note, with the ends of the piecewise sinusoidal segments 510a, 510b, 510c, and 510d aligned with the ends of the piecewise sinusoidal segments 530a, 530b, 530c, and 530d, the segments are formed out-of-phase, such as 180 degrees out of phase, as can be seen in comparing FIGS. 5A and 5B and more clearly in FIG. 5C, with an imaginary time or x-axis running from left to right in those FIGs. (note that FIG. 5B is shown on “top” of FIG. 5A in FIG. 5C).

FIG. 5C illustrates an assembled twisted pair interconnect 550 that is constructed with the piecewise sinusoidal segments 510a, 510b, 510c, and 510d and the piecewise sinusoidal segments 530a, 530b, 530c, and 530d, according to some example embodiments. In this regard, an insulating layer 555 (which, in some example embodiments, may be the base textile) may be disposed between the piecewise sinusoidal segments 510a, 510b, 510c, and 510d and the piecewise sinusoidal segments 530a, 530b, 530c, and 530d. According to some example embodiments, the insulating layer 555 may be formed of or from thermoplastic urethane (TPU). As such, the piecewise sinusoidal segments 510a, 510b, 510c, and 510d may be applied to a first side of the insulating layer 555 (e.g., the top side of the insulating layer 555 as shown in FIG. 5C) and the piecewise sinusoidal segments 530a, 530b, 530c, and 530d may be applied to a second side of the insulating layer 555 (e.g., the bottom side of the insulating layer 555 as shown in FIG. 5C). Application of the segments to the insulating layer 555 may involve removal of the segments from their respective substrates 500 and 520. In this regard, the piecewise sinusoidal segments 510a, 510b, 510c, and 510d may be positioned such that the ends of the segments overlap with the ends of the piecewise sinusoidal segments 530a, 530b, 530c, and 530d, as shown in FIG. 5C.

To electrically connect the segments, conductive vias may be included in the twisted pair interconnect 550. In this regard, a via may be the same as or similar to the feedthrough elements shown in FIGS. 2 and 3A to 3B and described above in both function and structure. In this regard, a via 551a, for example, may be positioned on the first end contact 511a of the piecewise sinusoidal segment 510a to permit electrical connection to the piecewise sinusoidal segment 510a on the bottom side of the insulating layer 555, as shown in FIG. 5C. Further, another via 551b may be included to form an electrical connection between the second end contact 512a of the piecewise sinusoidal segment 510a and the second end contact 531b of the piecewise sinusoidal segment 530b. Similarly, yet another via 551c may be included to form an electrical connection between a first end contact 511b of the piecewise sinusoidal segment 510b and the second end contact 532a of the piecewise sinusoidal segment 530a. As shown in FIG. 5C, similar vias may be installed between the segment ends, i.e. the associated end contacts to form connectivity across the twisted pair interconnect 550 assembly.

The assembled twisted pair interconnect 550 travels between the sides of the insulating layer 555 through the vias. As such, if each conductive path is followed through the twisted pair interconnect 550, the paths change sides of the insulating layer 555 as they move from one end to the other. Further, the paths overlap or crisscross along the length of the twisted pair interconnect 550. As a result of these characteristics, the individual interconnects of the twisted pair interconnect 550 are twisted together to form a twisted pair. As such, inductive coupling between the interconnects is increased, thereby providing substantial noise reduction benefits, while also maintaining a sinusoidal or serpentine shape/pattern to minimize stresses and strains on the individual interconnects due to movement of the base textile that the assembled twisted pair interconnect 550 may be affixed to. According to some example embodiments, other similar arrangements that approximate a twisted pair are also possible, such as, for example, arrangements with half-period or three-quarter-period segments, two-period or another integer-period segments, or combinations thereof, where the conductive path switches sides of the insulating layer 555 at an end of each segment.

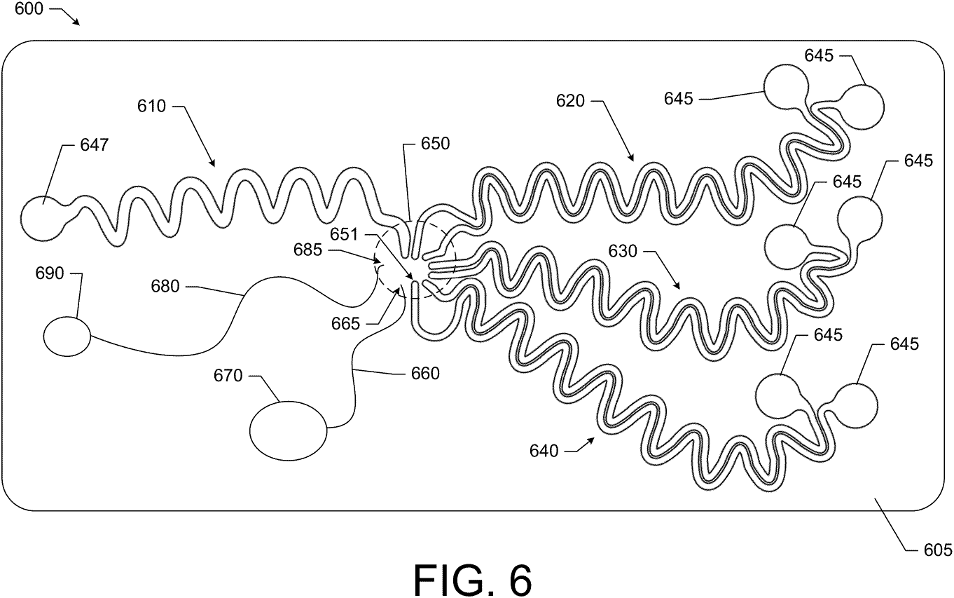

FIGS. 6, 7, 8A, and 8B will now be described to illustrate an example junction area and a tab assembly for connecting interconnects of a muscle activity sensor system 100 to a wireless circuit board 120 (e.g., a hard-to-soft connection), according to some example embodiments. In this regard, FIG. 6 illustrates an interconnect arrangement 600 including a base textile 605, an interconnect 610 (which may be used as a ground interconnect 160 connected to a ground connection 647, for example, on a user’s wrist), interconnect pairs 620, 630, and 640, a feedback connector 660, and a sensor connector 680. As shown, the interconnect 610, each of the interconnects 620, 630, and 640 (or pairs thereof), the feedback connector 660, and the sensor connector 680 all terminate in a junction area 650. More specifically, each of the interconnect pairs 620, 630, and 640 terminate at a respective interconnect junction contact 651 in the junction area 650, while the feedback connector 660 terminates at a feedback connector contact 665 and the sensor connector 680 terminates at a sensor connector contact 685 both terminate in the junction area 650.

Although they are shown following different paths in FIG. 6, the feedback connectors 660 and/or the sensor connectors 680 could be disposed along the same physical paths as, but electrically insulated from, the interconnects 610 and/or interconnect pairs 620. Specifically, in an example embodiment a conductive textile is used for the interconnects due its softness and ability to stretch. However, the electrical resistance of the conductive textiles is too high to be used for the feedback connectors 660. As a result, in an example embodiment where the feedback connectors 660 are disposed along the same physical paths as interconnect pairs 620, flexible printed circuit board material (e.g., KAPTON® film with copper electrical lines) is used for the feedback connectors 660, which are disposed along with (on or under) corresponding interconnect pairs 620, such that a given feedback connector 660 and corresponding interconnect pair 620 run along the same approximate physical path on a garment, giving support, but with some flexibility, to both the feedback connector 660 and the corresponding interconnect pair 620, while they remain electrically isolated from each other.

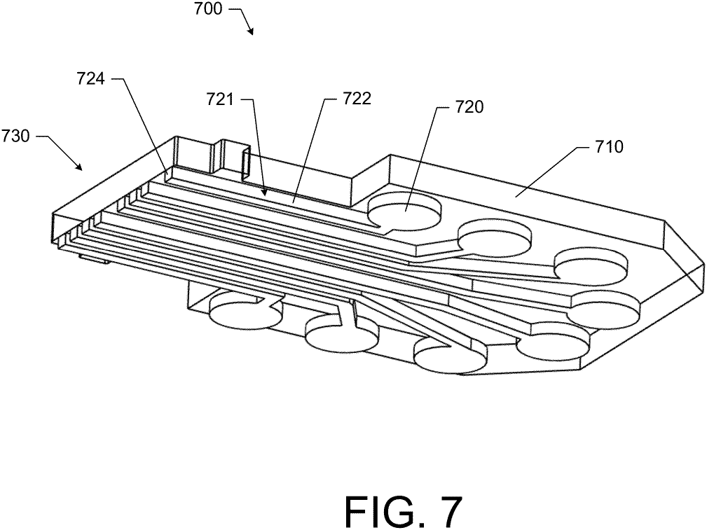

In an example embodiment, the interconnect junction contacts 651 may be terminal end portions of an associated interconnect pair (620, 630, 640) configured for connection of a corresponding muscle activity electrode 645 to a tab assembly/plug, as described in greater detail below with reference to FIG. 7. Similarly, the feedback connector contact 665 connects a feedback element 670 to the tab assembly/plug in the junction area 650, and the sensor connector contact 685 connects a sensor 690 to the tab assembly/plug in the junction area 650, as shown in FIG. 6. Although only one feedback element 670 (and its associated feedback connector 660 feedback connector contact 665) and only one sensor 690 (and its associated sensor connector 680 and sensor connector contact 685) are shown in FIG. 6, additional or alternative example embodiments are not limited thereto, in which case more than one feedback element 670 and/or sensor 690 (and their associated respective connectors connector contacts) may be included on the base textile 605. In addition, one or more of the muscle activity electrodes 645 (and their respective interconnect pairs 620, 630, or 640 and interconnect junction contacts 651) shown in FIG. 6 can be replaced with a feedback element 670 (and its respective feedback connector 660 and feedback connector contact 665).

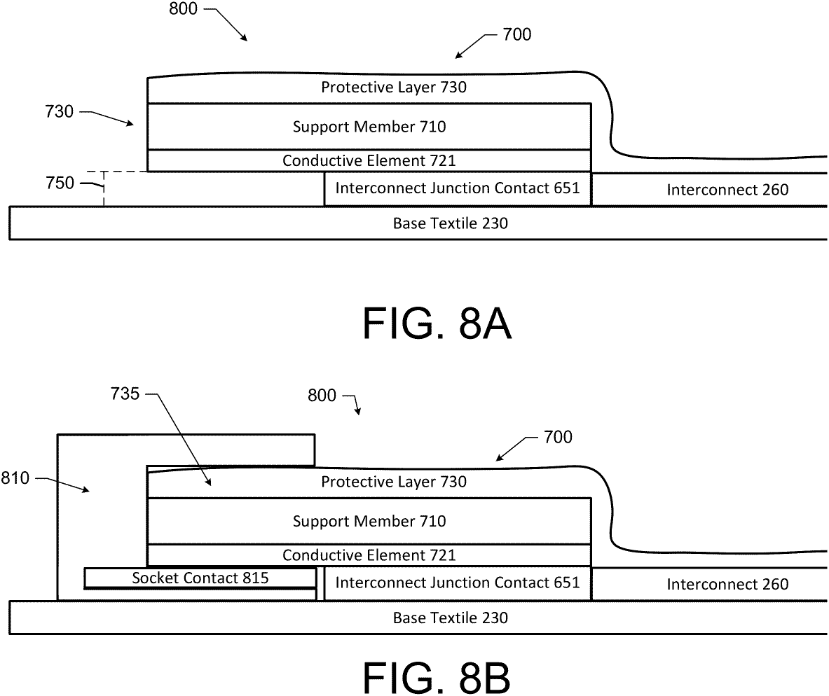

Referring now to FIG. 7, a tab assembly 700 according to an example embodiment is shown. The tab assembly 700 may include a number of conductive elements 721 affixed to a support member 710. Specifically, eight conductive elements 721 are shown in FIG. 7, but alternative example embodiments are not limited thereto. Each conductive element 721 may be associated with an interconnect 610 (FIG. 6), a single interconnect of an associated interconnect pair 620, 630, or 640 associated with a corresponding muscle activity electrode 645 (FIG. 6), a feedback connector 660 associated with a corresponding feedback element 670 (FIG. 6), or a sensor connector 680 associated with a corresponding sensor 690 (FIG. 6). In an example embodiment, the conductive elements 721 are formed of or from a conductive material (e.g., a metal, such as, copper, silver, aluminum, etc.). In this regard, each conductive element 721 may include a pad 720 configured to form an electrical connection with an interconnect junction contact 651, a feedback connector contact 665, or a sensor connector contact 685 (FIG. 6). Further, the conductive element 721 may include a trace 722 and a plug end 724. The trace 722 may be an electrical connector between the pad 720 and the plug end 724. The plug end 724 may be a contact point for the conductive element 721 to electrically connect the conductive element 721 (and thus the associated interconnect junction contact) to a socket contact, for example, within a circuit board socket on a circuit board (described in greater detail below with reference to FIG. 8B). As such, the plug end 724 may be one of a number of plug ends (e.g., eight plug ends, as shown in FIG. 7) associated with respective conductive elements, and the plug ends together with an end of the support member 710 may form a plug 735 (discussed below with reference to FIG. 8B).

The support member 710 may be formed of or from a flexible material, such as materials that are commonly used in flexible circuit boards. For example, the support member 710 may be formed by, of, or include a polyimide film (e.g., KAPTON® film). The support member 710 may have physical properties that permit some bending, but are also resilient to strain and other forces or stresses. As such, the conductive elements 721 that are affixed to the support member 710 may be protected due to these physical properties of the support member 710.

Now referring to FIG. 8A, with reference also back to FIG. 2, a cross-section view of a tab arrangement 800 according to some example embodiments is provided. The tab arrangement 800 may include the tab assembly 700 described above with reference to FIG. 7 and some additional components, coupled to an interconnect junction contact 651 (also shown in FIG. 6). In this regard, the base textile 230 may have the interconnect 260 affixed thereto, which may terminate at the interconnect junction contact 651. The tab assembly 700, including the support member 710 and the conductive element 721, may be affixed to the interconnect junction contact 651 via an adhesive, such as a conductive adhesive. More specifically, the pad 720 (FIG. 7) of the conductive element 721 may be adhered to the interconnect junction contact 651.

According to some example embodiments, a non-conductive adhesive may be used affix the conductive element 721 (and more specifically, the pad 720) to the interconnect junction contact 651. For example, a non-conductive heat lamination adhesive may be used. Rather unexpectedly, non-conductive adhesives used in this application are possible because the conductive knit fibers of the material used to form the interconnect junction contact 651 (e.g., conductive textiles including synthetic elastane fibers with a conductive coating or synthetic elastane fibers woven with conductive fiber) may penetrate through the adhesive to make electrical contact (e.g., direct physical contact) with the conductive element 721 and form a mechanical coupling. As such, according to some example embodiments, a patterned conductive adhesive layer may be unnecessary to couple the interconnect junction contact 651 to the conductive element 721 to mechanically connect the components and form an electrical connection when a non-conductive adhesive is used.

The tab assembly 700 may be positioned to have a portion that overhangs the base textile 230 and, due to the thickness of the interconnect 260 and the interconnect junction contact 651, a gap 750 may be formed between the overhanging portion and the base textile 230. This overhanging portion may be referred to as the plug 735 (FIG. 8B). Additionally, to protect the tab assembly 700 and the connection to the interconnect junction contact 651, the tab arrangement 800, and the muscle activity sensor, may include a protective layer 730. The protective layer 730 may be adhered over the tab assembly 700 and the interconnect 260. According to some example embodiments, the protective layer 730 may be disposed over each element of the muscle activity sensor (e.g., the electrode, the interconnect, and tab assembly). According to some example embodiments, the protective layer 730 may be formed of or from, or include a thermoplastic polyurethane or other suitable polymer that has an elastic property. With the tab assembly 700 affixed to the interconnect 260 and/or the interconnect junction contact 651, the tab assembly 700 may be permitted to bend and deflect without separating from the interconnect junction contact 651.

Referring now to FIG. 8B, a cross-section view of tab arrangement 800 engaged with a circuit board socket 810 is shown, according to some example embodiments. In this regard, the circuit board socket 810 may include a receiving cavity that receives the plug 735 such that a portion of the circuit board socket 810 slides into the gap 750 (FIG. 8A) between the conductive element 721 and the base textile 230. The circuit board socket 810 may include one or more socket contacts 815 that electrically connects to a respective conductive element 721 of the tab assembly 700. As such, via the circuit board socket 810, the electrical (e.g., EMG) signals initially detected by the electrodes of the muscle activity sensor may be provided to processing circuity (not shown) connected or coupled to the circuit board socket 810 or otherwise in communication with circuit board socket 810 for signal analysis.

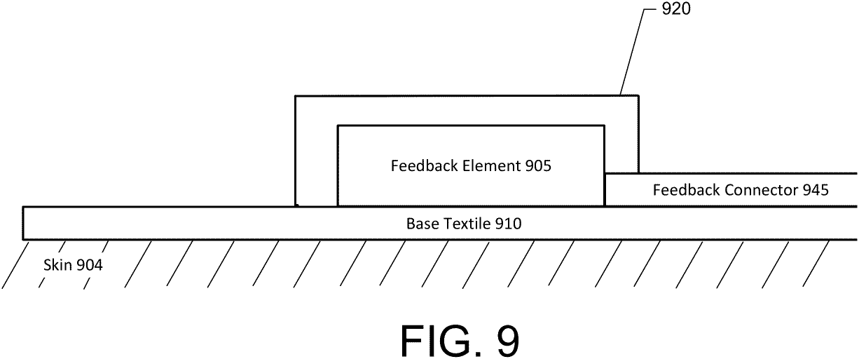

Feedback capabilities of a muscle activity sensor system according to some example embodiments will now be described in further detail with reference to FIGS. 9-12 (and also with reference again to FIG. 1).

Referring now to FIG. 9, which is a cross-sectional view illustrating the arrangement of a feedback element in a muscle activity sensor system according to an example embodiment, it can be seen that a feedback element 905 is disposed on (e.g., is adhered or otherwise affixed to), the base textile 910 (similar to the base textile 110 shown in FIG. 1). In one embodiment, the feedback element 905 is mounted on the base textile 910 using an adhesive (e.g., a hot-melt adhesive), lamination (e.g., heat lamination), or in another similar manner. Alternatively, the feedback element 905 may be attached to the base textile 910 by inserting the feedback element 905 into a pocket 920 or other similar enclosure disposed or formed on (or by/in) the base textile 910. A feedback connector 945 connects the feedback element 905 to a controller 120, e.g., a wireless controller 120 (FIG. 1).

In an example embodiment, the feedback element 905 is disposed on a side of the base textile 910 the opposite from the skin 904 of a user wearing a garment formed by or from the base textile 910, as shown in FIG. 9. However, in additional or alternative example embodiments, the feedback element 905 may be located on the other side of the base textile 910 shown in FIG. 9, that is, the feedback element 905 may be located on the skin 904 side of the garment, with or without any intervening layers (e.g., the pocket 920) between the feedback element 905 and the skin 904 of the user or person wearing the garment. In this case, the feedback connector 945 may also be located on the skin 904 side of the base textile 910, and/or the feedback connector 945 may penetrate through the base textile 910 from the skin 904 side to the opposite side thereof.

The feedback connector 945 may be formed from or of wire or flex connector, and may have very low resistance, particularly relative to a resistance of the interconnects 132. In one example embodiment, the resistance of the feedback connector 945 is about 5 ohms (Ω) or less. The feedback connector 945 may include two conductors, e.g., a positive and a negative (or ground) conductor, or may be a single conductor with a common ground plane or connection, as described in greater detail above with reference to FIG. 6. Additionally, as shown in FIG. 1, the feedback connector 945 (labeled “145” in FIG. 1) according to an example embodiment may follow a generally sinusoidal-shaped pattern along the base textile 110 (FIG. 1), and the feedback connector 945 may follow the same physical path as one of the interconnects 132 (FIG. 1), in which case the feedback connector is electrically insulated from the associated interconnect.

Still referring to FIG. 9, the feedback element 905 may be a haptic and/or tactile feedback element 905, which transmits a physical response to select sensory nerve locations of a user or wearer of a garment having the muscle sensor activity system 100 (FIG. 1) included therein. More particularly, multiple tactile feedback elements 905 may be located specifically where sensory nerve re-innervation surgery has been performed or where sensory nerves have re-innervated naturally on injured or prosthetic patients, to create or enhance the patient’s sense of touch, to enhance identification and control of objects, or to alert the patient to various properties of the object or the surrounding environment.

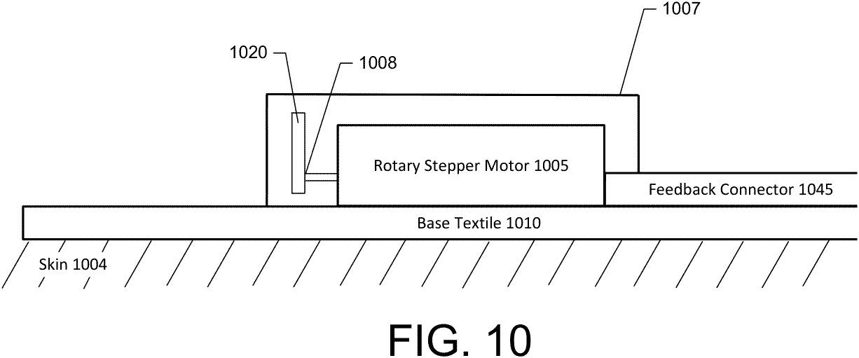

A cross-sectional view of one specific example embodiment of a haptic feedback element is shown in FIG. 10. Specifically, the haptic feedback element shown in FIG. 10 is a motor and, more particularly, is a rotary stepper motor 1005 disposed in a rigid (or semi-rigid) enclosure 1007 on a base textile 1010. A feedback connector 1045 connects the rotary stepper motor 1005 to a controller, e.g., a wireless controller, which will be described in greater detail below. In an example embodiment, the rotary stepper motor 1005 drives a shaft 1008, which in turn drives a rotating element 1020, which may be an eccentric rotating element or mass (e.g., a disc) 1020 that presses on the skin of a user or wearer with varying force, depending on a speed and/or position of the rotary stepper motor 1005. As described above with reference to FIG. 9, the rotary stepper motor 1005 may be located on a side of the base textile 1010 opposite the skin 1004 of a user or wearer of a garment formed by or from the base textile 1010, as shown in FIG. 10, but the rotary stepper motor 1005 may alternatively be located on the other side of the base textile 1010, i.e. on the skin 1004 side of the garment.

As the rotary stepper motor 1005 rotates the shaft 1008, the rotating element 1020 rotates to intermittently press against the skin 1004 of a user or wearer of a garment including the muscle activity sensor system 100 to provide haptic feedback to the user. More specifically, the rotary stepper motor 1005 may operate at a relatively high speed to produce a rapid, vibrating sensation (e.g., a perceived “buzz”) to the user, or at lower speeds to impart a different sensation. In an example embodiment, the rotational angle of the rotary stepper motor 1005 may also be adjusted, to change the perceived intensity of the sensation, for example. Additionally, the rotary stepper motor 1005 may be used at very low frequencies, or in a binary or “on-off” manner, to provide a one-time (or constant) feedback sensation to the user, such that, when the rotary stepper motor 1005 is energized, it positions the eccentric rotating element 1020 to press against the user’s skin and, when the rotary stepper motor 1005 is turned off, the eccentric rotating element 1020 is rotated to not press against the user’s skin (or vice versa).

While a specific mechanical haptic feedback device, i.e. the rotary stepper motor 1005, is shown in FIG. 10, additional or alternative example embodiments are not limited thereto. For example, different mechanical feedback elements 905 (FIG. 9), include, but are not limited to, different types of motors (e.g., vibration or “vibromotors”), or different implementations of force by such motors. Specifically, a motor could be used to exert pressure on the skin by winding up and/or relaxing an elastic band attached to the garment to “bunch” up the garment against the skin. Other alternative ways to provide forces for haptic feedback include pneumatic (e.g., air flow or vortices), ultrasound, and electrostatic.

The feedback elements 905 in other example embodiments may include other nonmechanical feedback, as well. For example, the feedback element 905 may provide thermal e.g., hot and/or cold) feedback, such as with a thermoelectric device/actuator the same or similar to the one shown and described in U.S. Pat. No. 11,227,988, issued Jan. 18, 2022 and titled “Fast-Rate Thermoelectric Device,” which is incorporated herein by reference in its entirety.

In other example embodiments, the feedback elements 905 may provide still different types of feedback, including visual (e.g., light), audible (e.g., sound), electrical (including for muscle or nerve stimulation, such as in patients who have had sensory nerve reinnervation surgery), piezoelectric/ultrasound vibrations, etc.

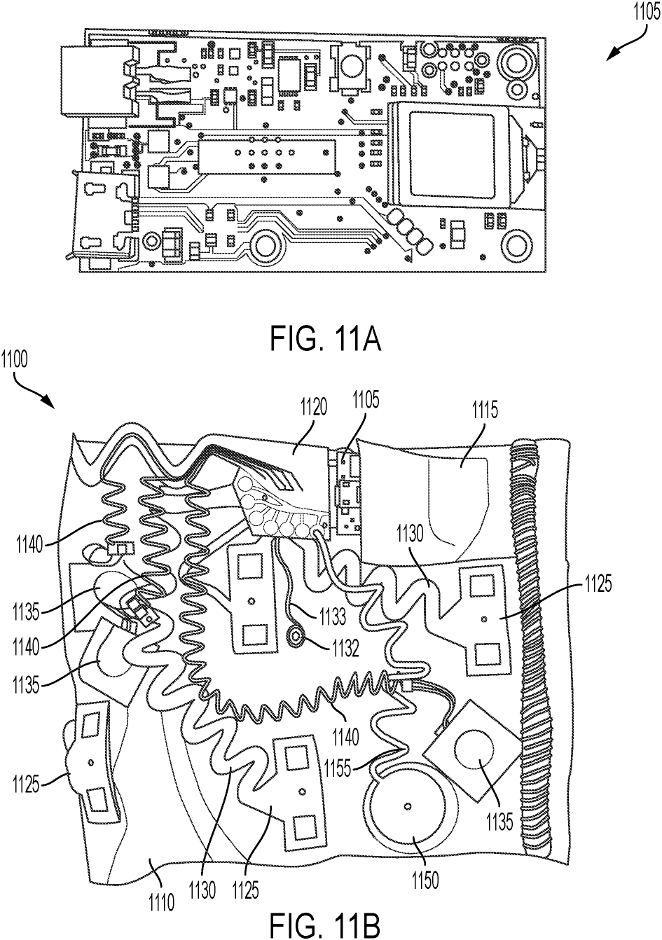

As mentioned above, a controller 120 (FIG. 1) implements the feedback and/or sensing capabilities described herein, as will now be described in further detail with reference to FIGS. 11 - 13.

FIG. 11A illustrates a controller according to an example embodiment, and FIG. 11B illustrates a muscle activity sensor system, including the wireless controller shown in FIG. 11A, disposed in a garment according to an example embodiment.

As shown in FIGS. 11A and 11B, a muscle activity sensor system 1100 includes a controller 1105, which may be a wireless controller 1105, disposed in a garment 1110. In an example embodiment, the garment 1110 is a sleeve (or a portion thereof) that is worn on the arm of a user (as shown in FIG. 1), but alternative example embodiments are not limited thereto. As shown in FIG. 11B, the wireless controller 1105 is disposed in a pocket (or sleeve) 1115 of the garment 1110. The wireless controller 1105 is connected to the other components of the system 1100 by a tab assembly 1120, described in greater detail above with reference to FIG. 7 (tab assembly 700). Specifically, in the example embodiment shown in FIG. 11B, the wireless controller 1105 is connected, through the tab assembly 1120, to muscle activity electrodes 1125 via interconnect pairs 1130, to sensors 1132 via sensor connectors 1133, and to feedback elements 1135 via feedback connectors 1140. The feedback elements 1135 shown in FIG. 11B are vibromotors 1135, but it will be understood that alternative example embodiments are not limited thereto. As can also be seen in FIG. 11B, a ground connection 1150 is connected to the tab assembly 1120 by an interconnect 1155.

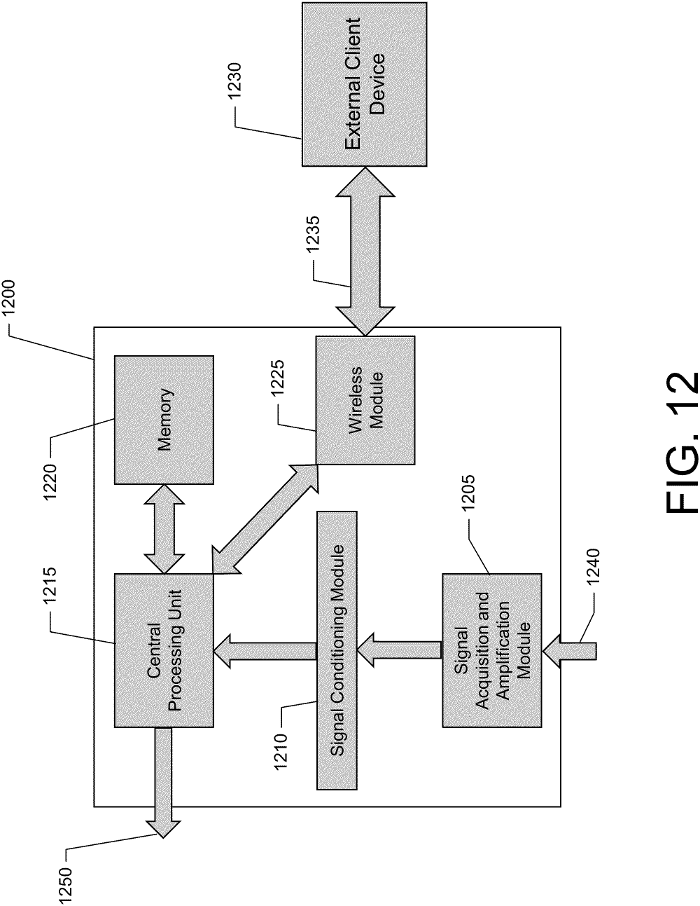

FIG. 12 is a block diagram of a controller 1200, which may be a wireless controller 1200, according to an example embodiment, in which the wireless controller 1200 includes a signal acquisition and amplification module 1205, which connects via an optional signal conditioning module 1210 to a central processing unit (CPU) 1215. A memory 1220 is connected to the CPU 1215, as is a wireless module 1225. The wireless controller 1200 communicates with an external client device 1230 via an interface 1235. In an example embodiment, the external client device 1230 may be a laptop computer, a cellular telephone, a prosthetic limb, an exoskeleton, a robot system, or the like. The wireless controller 1200 may also include (or be connected to) other components (not shown), such as for supplying power (e.g., a battery or micro-USB connection) and other functions.

In one example embodiment, the interface 1235 is a wire (e.g., the wired connection 122 shown in FIG. 1). In another example embodiment, the interface 1235 is wireless, in which case the wired connection 122 is not needed, and the wireless controller 1200 communicates with the external client device 1230 via the wireless interface 1235. In this case, the wireless interface 1235 may utilize a short range wireless technology such as BLUETOOTH® and, more particularly, in an example embodiment, the wireless module 1225 is a BLUETOOTH® Low Energy (BLE) module, although additional example embodiments are not limited thereto. For example, alternative example embodiments may utilize other wireless technologies, such as other radio and radio-frequency technologies, wireless local area networking products such WIFI® or WI-FI®, and electromagnetic induction, etc.

The signal acquisition and amplification module 1205 of the wireless controller 1200 receives an input signal 1240 from the garment 1110 (FIG. 11B). As described above in greater detail with reference to FIG. 1, described above, the input signal 1240 may include information from the muscle activity electrodes 130 (e.g., from an electrical or EMG signal) and/or information from the sensors 150 (e.g., a sensor signal), such that the wireless controller 1200 (and/or the external client device 1230) can process the muscle activity and/or sensor information to generate, among other things, a feedback signal 1250 (e.g., a stimulation feedback signal 1250), as will now be described in further detail.

Still referring to FIG. 12, in an example embodiment, the signal acquisition and amplification module 1205 is an INTAN TECHNOLOGIES® amplifier integrated circuit (IC), although additional example embodiments are not limited thereto. The signal acquisition and amplification module 1205 passes the input signal 1240 to the CPU 1215 (via the signal conditioning module 1210, when present). The CPU 1215, which, according to a non-limiting, example embodiment is a 32-bit ARM® Cortex-M4 CPU running at up to 64 megahertz (MHz), in connected to, e.g., is in communication with (or in some embodiments, having embedded therein), the memory 1220. The memory 1220 may include flash memory and/or random access memory (RAM). The CPU 1215 processes the input signal 1240 (having been amplified and in some cases conditioned) to generate the stimulation feedback signal 1250, which is sent to the feedback elements 1135 via the feedback connectors 1140 shown in FIG. 11B.

As shown in FIG. 12, the CPU 1215 is also in communication with (e.g., is connected to) the wireless module 1225, which enables the wireless controller 1200 to transfer the electrode and/or sensor information between the CPU 1215 and the external client device 1230. Thus, the external client device 1230 may process the electrode and/or sensor information in the input signal 1240 to supplement, or even generate, the stimulation feedback signal 1250. Regardless of whether the wireless controller 1200 or the external client device 1230 (or both of them) process the electrode and/or sensor information, the basic flow of information in such processing will now be described in further detail with reference to FIG. 13.

As shown in FIG. 13, which illustrates a flow of information in a wireless controller and/or external client device according to some example embodiments, in a biosignal acquisition and transmission process 1300, biosignal (e.g., EMG) information is received from electrodes 1125 via interconnects 1155 (FIG. 11B) in step 1302. In step 1304, the biosignal is acquired and amplified. In step 1306, the biosignal is optionally conditioned, which may include, but is not limited to, low pass filtering, high pass filtering, signal compression, re-sampling, format conversion, integration, differentiation, addition, and subtraction. The biosignal is packaged for transmission in step 1308 and, in step 1310, the amplified, optionally conditioned, and packaged biosignal is transmitted to the external client device 1230 and/or the CPU 1215 (FIG. 12).

In a biosignal control process 1320, step 1322 includes receiving an input command from the external client device 1230 and/or the CPU 1215 (FIG. 12), which selectively enables and disables channels for transmitting the biosignal (step 1324).

Still referring to FIG. 13, a sensory control and feedback process 1330 includes, in step 1332, receiving sensor signals/information related to, indicative of, or based upon of a condition or parameter at or near (e.g., proximate to) the sensor and/or base textile or the environment of a user or wearer of the garment (such as pressure, temperature, moisture, oxygen level, etc., as described above with reference to the sensors 150 shown in FIG. 1) and, in step 1334, analyzing and mapping the incoming sensor signals to their respective stimulation feedback signals 1250 (FIG. 12). In step 1336, the stimulation feedback signal(s) 1250 are generated and, in step 1338, are transmitted to their associated feedback elements 1135 (FIG. 11B). Specifically, for example, a temperature sensor 150 (FIG. 1) may sense an ambient temperature and provide an associated temperature sensor signal (input signal 1240) to the wireless controller 1200 (FIG. 12) (step 1332). The wireless controller 1200 processes the temperature signal (step 1334) to determine the ambient temperature and, if the ambient temperature is above a predefined threshold value, generate and transmit (step 1336) a stimulation feedback signal 1250 to an associated feedback element 1135, causing the feedback element 1135 to heat up, alerting the user wearing the garment 1110 to a high temperature environment.

The sensor system 1100 and, in particular, the wireless controller (1105, 1200) as described above with reference to FIGS. 11B, 12, and 13, is configured to sense, receive, process, and output various signals related to sensors (e.g., temperature), electrodes (electrical or EMG signals from muscles), and feedback elements. In one specific example embodiment, the sensor system 1100 is capable of collecting EMG data from up to 10 separate electrodes 1125 and broadcasting the associated biosignals (EMG information) via BLUETOOTH® at 400 Hertz (Hz) or higher. The system 1100 also supports BLUETOOTH® control 4 or more feedback elements 1135. It will be understood that alternative example embodiments are not limited to the configuration and capabilities described above; rather, that the number and locations of the components shown on the garment 1110 (FIG. 11B), including the wireless controller 1105, the electrodes 1125, and/or the feedback elements 113, may be varied to suit the needs of a particular user (wearer) of the garment 1110.

More generally speaking, the non-limiting, example embodiments presented herein are provided as examples and therefore the disclosure is not to be limited to the specific embodiments disclosed. Modifications and other embodiments are intended to be included within the scope of the appended claims. Moreover, although the foregoing descriptions and the associated drawings describe example embodiments in the context of certain example combinations of elements and/or functions, different combinations of elements and/or functions may be used to form alternative embodiments. In this regard, for example, different combinations of elements and/or functions other than those explicitly described above are also contemplated. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments.

What is claimed is:

1. A system for muscle activity sensing and feedback, the system comprising:
a base textile;
an electrode coupled to the base textile;
a sensor coupled to the base textile;
a controller coupled to the base textile; and
a feedback element coupled to the base textile and in communication with the controller, wherein
the feedback element:
receives a feedback signal from the controller, and
imparts feedback to a user based on at least one of an electrical signal from the electrode and a sensor signal from the sensor.

2. The system of claim 1, wherein the feedback is haptic.

3. The system of claim 2, wherein the feedback element comprises a motor.

4. The system of claim 1, wherein the feedback is thermal.

5. The system of claim 4, wherein in the feedback element comprises a thermoelectric device.

6. The system of claim 1, wherein the feedback is visual, audible, or electrical.

7. The system of claim 1, wherein the controller is in wireless communication with at least one of the electrode and the feedback element.

8. The system of claim 1, wherein the controller is in wireless communication with an external client device.

9. The system of claim 1, further comprising a tab assembly coupled to the base textile and coupled between the controller and the electrode, the sensor, and the feedback element, wherein the controller comprises:
a signal acquisition and amplification module; and
a central processing unit connected to the signal acquisition and amplification module, wherein
the signal acquisition and amplification module receives, via the tab assembly, the at least one of the electrical signal from the electrode and the sensor signal from the sensor and amplifies the at least one of the electrical signal and the sensor signal, and
the central processing unit processes the amplified at least one of the electrical signal and the sensor signal, generates the feedback signal based on the amplified at least one of the electrical signal and the sensor signal, and provides the feedback signal to the feedback element via the tab assembly.

10. The system of claim 9, wherein
the controller further comprises a wireless module connected to the central processing unit, and
the central processing unit communicates with an external client device via the wireless module.

11. A system for muscle activity sensing and feedback, the system comprising:
a base textile configured to apply a compression force against a dermal surface of a user;
an electrode coupled to the base textile and configured to receive an electrical signal associated with muscle activity of the user;
a sensor coupled to the base textile and configured to sense a parameter associated with a condition of either the base textile or an environment near the base textile and to generate a sensor signal based on the sensed parameter;
a controller coupled to the base textile and configured to receive at least one of the electrical signal from the electrode and the sensor signal from the sensor, to analyze the at least one of the electric signal and the sensor signal, and to generate a feedback signal; and
a feedback element coupled to the base textile and in communication with the controller, the feedback element being configured to receive the feedback signal from the controller and to impart feedback to the user based on the at least one of the electrical signal from the electrode and the sensor signal from the sensor.

12. The system of claim 11, wherein the feedback is haptic.

13. The system of claim 12, wherein the feedback element comprises a motor.

14. The system of claim 11, wherein the feedback is thermal.

15. The system of claim 14, wherein in the feedback element comprises a thermoelectric device.

16. The system of claim 11, wherein the feedback is visual, audible, or electrical.

17. The system of claim 11, wherein the controller is further configured to be in wireless communication with at least one of the electrode and the feedback element.

18. The system of claim 11, wherein the controller is further configured to be in wireless communication with an external client device.

19. The system of claim 11, further comprising a tab assembly coupled to the base textile and coupled between the controller and the electrode, the sensor, and the feedback element, wherein the controller comprises:
a signal acquisition and amplification module configured to receive, via the tab assembly, the at least one of the electrical signal from the electrode and the sensor signal from the sensor and amplify the at least one of the electrical signal and the sensor signal; and
a central processing unit connected to the signal acquisition and amplification module, the central processing unit being configured to processes the amplified at least one of the electrical signal and the sensor signal, to generate the feedback signal based on the amplified at least one of the electrical signal and the sensor signal, and to provide the feedback signal to the feedback element via the tab assembly.

20. The system of claim 19, wherein the controller further comprises a wireless module connected to the central processing unit and configured to communicate with an external client device.
